(19) United States
(12) Patent Application Publication
(10) Pub. No.: US 2023/0235279 A1
(43) Pub. Date: Jul. 27, 2023

(54) AMNIOTIC-LIKE EPITHELIAL CELL GENERATION

(71) Applicant: THE BABRAHAM INSTITUTE, Cambridge (GB)

(72) Inventors: Maria ROSTOVSKAYA, Cambridge (GB); Peter RUGG-GUNN, Cambridge (GB)

(21) Appl. No.: 17/927,968

(22) PCT Filed: May 28, 2021
(86) PCT No.: PCT/GB2021/051321

§ 371 Date: Nov. 28, 2022

(30) Foreign Application Priority Data
May 29, 2020 (GB) 2008119.6

(51) Int. Cl. C12N 5/073; A61K 35/36

(52) U.S. Cl. CPC C12N 5/0605; A61K 35/36; C12N 2501/15; C12N 2501/727; C12N 2506/45

(57) ABSTRACT

The present invention relates to a reliable method for producing amniotic-like epithelial cells, using a new methodology. The invention also relates to a composition and the use of said composition comprising amniotic-like epithelial cells or a preparation derived therefrom. Said cells may have particular utility in regenerative medicine, research and/or cosmetic preparations.

The present invention relates to a method for producing amniotic-like epithelial cells, using a new methodology. The invention also relates to a composition and the use of said composition comprising amniotic-like epithelial cells prepared according to the method disclosed. Such cells may have a particular utility in research, and therapy including regenerative medicine and for cosmetic preparations. Alternatively, compositions derived from the cells, such as membranes, cells in matrices or scaffolds and/or cell extracts may be used. Such amniotic like epithelial cells exhibit low expression levels of human leukocyte antigens (including HLA-A, HLA-B, and HLA-C and HLA-DR), which are key antigens involved in recipient rejection, meaning that allogenic cell transfer is possible. They are, therefore, a desirable cell phenotype for use in therapy. Optionally, the cells can be further differentiated in vitro.

### BACKGROUND TO THE INVENTION

The amnion is an extraembryonic epithelial tissue that forms a membrane surrounding the developing embryo. In primates including humans, amniotic epithelium originates from pluripotent epiblast during implantation. During post-implantation development, amnion functions to mechanically protect the embryo, produce growth factors, cytokines and hormones, maintain the pH in amniotic fluid. Furthermore, in contrast to rodents, early nascent amnion in primates was suggested as a source of primordial germ cells (PGC), secreting growth factors for their differentiation in an autocrine fashion, therefore amnion serves as a unique self-organising centre of PGC specification.

Amniotic membrane is an attractive source for tissue engineering and regenerative therapies, because of its anti-inflammatory and immunomodulatory properties, ability to induce epithelialisation, and lack of tumorigenicity and ethical issues in clinical application. Amniotic membrane collected from term placenta has been successfully applied in patients for ocular surface reconstruction and treatments of burns and wounds. Despite their fundamental and clinical importance, the properties of amnion cells remain poorly characterised and the current approaches of their clinical application suffer from very limited expansion of amniotic epithelial cells in vitro. There is thus a pressing need of improved sources of human amniotic epithelial cells, and new methods to generate and expand populations of amniotic epithelial cells in vitro.

Amniotic epithelial cells (AECs) are extracted from the lining of the inner membrane of the term placenta. Amniotic epithelial cells show low immunogenicity, in addition to immunomodulatory and anti-inflammatory behaviours. Because of these qualities, AECs have been proposed for and indeed used in regenerative medicine. However, since there are regional restrictions on the ability of clinicians to use placental material, particularly in the US, and there are technical limitations on propagating cells from placental material, alternative sources of AECs are desirable. Further, cells from the placenta risk the transmission of infectious diseases and bacterial contamination.

Therefore it is desirable to be able to generate a stable and robust source of AECs.

Regenerative medicine involves the generation of healthy cells to replace diseased cells, or to produce factors stimulating endogenous regenerative mechanisms. Stem cells can be guided into becoming specific cells that can be used to regenerate and repair diseased or damaged tissues in people. Most regenerative medicines require the use of pluripotent stem cells, such as embryonic stem cells (ESCs) or induced pluripotent stem cells (iPSCs), with the latter being cells generated by the use of particular reprograming factors or conditions on non-pluripotent cell types.

Pluripotent cells can give rise to all of the cell types that make up the body; embryonic stem cells are considered pluripotent. Pluripotency is defined as the capacity of single cells to produce differentiated progeny of the three principal germ layers and the germline. In the human embryo, pluripotency is a characteristic of epiblast cells from the early pre-implantation stage until lineage specification during gastrulation, lasting for at least 10 days. During this window, the epiblast cells progress through several distinct developmental phases and therefore, pluripotency is a generic property of cells with different identities. As such, two extreme states of pluripotency have been defined: naïve cells correspond to the early pre-implantation epiblast and primed cells are reminiscent of the pre-gastrulation stage.

There have been many attempts to capture and evaluate human naïve pluripotency, with some studies successfully establishing multiple protocols to generate human naïve pluripotent stem cells by direct derivation from embryos, reprogramming from somatic cells or conversion from conventional primed pluripotent stem cells. Several groups have shown conversion of primed state human pluripotent stem cells to the naïve state, using overexpression of transgenes, or by treatment with specific media (Theunissen et al (2014) Cell Stem Cell, 15(4): 471-487; Takashima et al (2014) Cell, 158(6): 1254-1269; Guo et al (2017) Development, 144(15): 2748-2763). Shiozawa et al. has shown that using transgenes you can convert primed state embryonic stem cells from common marmoset (monkey) to the naïve state (Shiozawa et al. Stem Cells Dev. 2020, https://doi.org/10.1089/scd.2019.0259). Guo et al. and Boroviak et al. provided a useful model for mechanistic studies of pluripotency regulation and lineage differentiation by establishing human and mouse naïve pluripotent stem cells from the epiblasts of preimplantation blastocysts (Guo et al (2016) Stem Cell Reports, 6(4): 437-446.). Naïve pluripotency only exists for a short period of time during mammalian embryonic development (Nakamura et al (2016) Nature, 537(7618): 57-62; Boroviak et al (2014) Nat Cell Biol, 16(6): 516-528). Naïve cells have an unlimited self-renewal capacity when grown under appropriate conditions and are able to differentiate into tissues of all three germ layers in vitro.

An experimental in vitro system for conversion of human naïve pluripotent stem cells to the primed state has been recently established (Rostovskaya et al (2019) Development, 146(7): dev172916). The conversion of naïve to primed pluripotent stem cells, also called capacitation, or formative transition, has been shown to recapitulate features of peri-implantation progression of embryonic epiblast.

Naïve and primed hPSC have distinct signalling requirements for sustained self-renewal in vitro (Theunissen et al (2014) Cell Stem Cell, 15(4): 471-487; Takashima et al (2014) Cell, 158(6): 1254-1269). The maintenance of naïve hPSC requires the inhibition of the mitogen-activated protein kinase (MAPK) pathway, whereas the propagation of primed hPSC depends upon the activity of this pathway (Vallier et al (2005) JCS, 118: 4495-4509). The mitogen-activated protein kinase (MAPK) pathway is a chain of proteins in a cell which results in the communication of a signal from a receptor on the surface of the cell to the DNA in the nucleus of the cell. The proteins convert extracellular stimuli into a wide range of cellular responses. All eukaryotic cells possess complex branched highly pleiotropic MAPK pathways. These co-ordinately regulate gene expression, mitosis, metabolism, motility, survival, apoptosis and differentiation. The central protein within these pathways are protein Ser/Thr kinases called mitogen-activated protein kinases (MAPK). The dysregulated signalling of the MAPK proteins in the pathway can result in excessive cell proliferation and survival, which may play a role in specific malignancies.

The TGF beta signalling pathway is also involved in many of the cells processes in both embryonic development and adult organisms. These cellular processes can include cell growth, cell differentiation, apoptosis and cellular homeostasis. The TGF beta superfamily of ligands includes Growth and differentiation factors (GDFs), Anti-müllerian hormone (AMH), Nodal and TGFβs, as well as others. Signalling begins with the binding of a ligand to a TGF beta type II receptor. This receptor recruits and phosphorylates a type I receptor. The type I receptor will then phosphorylate receptor-regulated SMADs which can bind and form a complex with coSMAD that accumulates in the nucleus. This complex accumulation can act as transcription factors and participates in the regulation of target gene expression.

None of the previous work performed with pluripotent stem cells, as far as the present inventors are aware, has resulted in the purposive differentiation of cells into amnion-like epithelial cells recapitulating their developmental pathway. Work has been performed in analysing early embryogenesis to determine where and when the amnion arises during development (Luckett (1975) Dev Dynam 144(2): 149-167; Enders et al (1986) Am J Anat 177(2): 161-185; Nakamura et al (2016) Nature 537(7618): 57-62; The Virtual Human Embryo Atlas) concluding that amnion emerges during the peri-implantation period. Pluripotent stem cells have been used in attempts to identify whether naïve or primed pluripotent stem cells are the predecessors of potential amnion fate. Such earlier works include Guo et al (Guo et al (2021) Cell Stem Cell doi: 10.1016/j.stem.2021.02.025) and Io et al (Io et al (2021) Cell Stem Cell doi: 10.1016/j.stem.2021.03.013). However, both of these works did not use the appropriate starting pluripotent stem cell state (which is in between naïve and primed states that corresponds to peri-implantation embryonic state). The authors of both works conclude that only primed cells are capable of differentiation into cells that express putative markers of amnion - these markers are BAMB1, ISL1, ITGB6, SEMA3C and IGFBP3 - none of which are established as specific markers for amnion cells. Moreover, their work did not analyse the cells produced in terms of the appearance or development of any form of epithelium or three dimensional cavitating structures. Further, others have experimented upon the physical environment of primed pluripotent stem cells to see if this is of use in determining the development of tissues forming the amnion. WO2018/106997 discloses methods for deriving amnion tissues from stem cells using scaffolds and devices, these act as a biomimetic post-implantation niche. Such devices aim to recapitulate amniogenesis shortly after implantation (embryogenesis). This is further described in Shao et al (Nat Mater 2017 16(4); 419-425). In all three cases, research has been done using primed pluripotent stem cells and none of these works were performed using hPSC in capacitating conditions. Further, in all three cases a BMP-signalling dependent in vitro differentiation pathway is investigated.

The present inventors have devised a novel method to derive amnion-like epithelial cells with high efficiency from pluripotent stem cells, which recapitulates developmental events in the embryo, permitting the establishment of a robust and effective source of amnion-like epithelial cells that will be of great use therapeutically and for research purposes.

### SUMMARY OF THE INVENTION

The present invention provides a method for differentiating pluripotent stem cells into amniotic-like epithelial cells, said method comprising culturing said cells with an inhibitor of the MAPK pathway and an inhibitor of the TGF pathway.

Thus, the present invention is a method which involves the culturing of pluripotent stem cells in particular conditions which permits the differentiation of the pluripotent stem cells into amniotic-like epithelial cells. The method can therefore be described as ex vivo or in vitro, since the method takes place outside the human or animal body.

The method may comprise amniotic-like epithelial cells that form a continuous layer of cells. The continuous layer of cells may form a membrane or a 3D structure. The cells may be human cells, or animal cells.

Therefore, the amniotic-like epithelial cells of the invention can be observed to form a continuous layer of cells once cultured under appropriate conditions. Alternatively put, the amniotic-like cells form an epithelium.

The method of the present invention involves the culturing of a pluripotent stem cell. Said pluripotent stem cell may be any suitable pluripotent stem cell. The cells may be isolated from an embryo, isolated from a parthenote, or taken from an established embryonic stem line, or be an induced pluripotent stem cell. It is preferred that the pluripotent stem cell is obtained without destruction of an embryo.

Optionally, the pluripotent stem cells cultured according to the method of the present invention are any one or more of:
a. naïve pluripotent stem cells;
b. naïve pluripotent stem cells cultured under capacitating conditions;
c. primed pluripotent stem cells cultured under conditions reverting them to naïve pluripotent stem cells; and/or
d. pluripotent stem cells representing intermediate states between the naïve and the primed pluripotent states.

The cells of section (d) optionally include, but are not limited to, formative cells, cells that correspond to the intermediates during the formative transition.

Those skilled in the art will appreciate that the term “pluripotent” actually covers a variety of cell types between naïve cells and primed cells.

Optionally, the pluripotent stem cell according to the present invention is any pluripotent stem cell with the exception of a primed pluripotent stem cell.

The method of the present invention may also optionally include culturing the pluripotent stem cell with a BMP inhibitor.

The method of the present invention comprises the use of a MAPK pathway inhibitor. This MAPK pathway inhibitor can be any suitable inhibitor of any member of this pathway, and those skilled in the art will be aware of suitable inhibitors. The inhibitor can be direct inhibitor, i.e. have a direct effect on the MAPK pathway component, or be indirect, for example induce an inhibiting effect within the cell. Optionally the MAPK pathway inhibitor may be a chemical inhibitor, neutralising antibody, aptamer, ligand trap, antisense nucleotide, protein inhibitor, and engineered peptide targeting any one from the list comprising: receptor tyrosine kinases, Ras, Src, Raf, MEK½, p38 MAP kinases, ERK½; or activators or agonists of AKT and PI3K. Optionally, the MAPK pathway inhibitor may be an indirect inhibitor of the MAPK pathway. For example, the MAPK inhibitor could be a compound or agent which induces expression of components required for gene knockdown or knockout of a MAPK pathway component. Examples of such a system may be DNA or RNA editing inducible programmable nucleases, notably the CRISPR/Cas9 system, small interfering RNAs, epigenetic editing systems.

It may be preferred that the inhibitor targets (directly or indirectly) any component of the MAPK/ERK pathway, such as RAS, RAF, MEK and/or ERK (also called MAPK). In one embodiment, the inhibitor may target MEK (MEK1 and/or MEK2). In one embodiment, the inhibitor may target MAPK (ERK½).

The method of the present invention comprises the use of a TGF pathway inhibitor. This TGF pathway inhibitor can be any suitable inhibitor of any pathway member, and those skilled in the art will be aware of suitable inhibitors. The inhibitor can be direct inhibitor, i.e. have a direct effect on the TGF pathway component, or be indirect and for example to induce an inhibiting effect within the cell. Optionally the TGF pathway inhibitor includes a chemical inhibitor, neutralising antibody, ligand trap, aptamer, antisense nucleotide, protein inhibitor, engineered peptide targeting any one from the list comprising: ligands TGF beta, Activin, Nodal; TGF beta type I receptors TGFBR1, ACVR1, ACVRL1, ACVR1B, ACVR1C; TGF beta type II receptors TGFBR2, ACVR2A, ACVR2B; signal transducers Smad2, Smad3, Smad4; TGF ligand processing enzyme furin. Optionally, the TGF pathway inhibitor may be an indirect inhibitor of the TGF pathway. For example, the TGF inhibitor could be a compound or agent which induces expression of components required for gene knockdown or knockout of a TGF pathway component. Examples of such a system may be DNA or RNA editing inducible programmable nucleases, notably the CRISPR/Cas9 system, small interfering RNAs, epigenetic editing systems.

It may be preferred that the inhibitor targets (directly or indirectly) any component of the TGF beta pathway, such as TGF beta type I receptors TGFBR1, ACVR1, ACVRL1, ACVR1B, ACVR1C; TGF beta type II receptors TGFBR2, ACVR2A, ACVR2B; signal transducers SMAD2, SMAD3, SMAD4; TGF ligand processing enzyme furin. In one embodiment, the inhibitor may target TGF beta type I and/or TGF beta type II receptors. It may be preferred that the inhibitor is capable of inhibiting SMAD signalling but not BMP signalling.

Optionally, the method of the present invention may comprise the use of a BMP inhibitor. This inhibitor is additional to those described above. This BMP inhibitor may be any suitable inhibitor, and those skilled in the art will be aware of suitable inhibitors. The inhibitor can be direct BMP inhibitor, or be indirect, for example induce an inhibiting effect within the cell. Optionally the BMP inhibitor includes can be a chemical inhibitor, neutralising antibody, ligand trap, aptamer, antisense nucleotide, protein inhibitor, engineered peptide targeting any one from the list comprising: ligands BMP2, BMP4, BMP7; BMP type I receptors BMPRIA, BMPRIB; BMP type II receptor BMPR2, Smad1, Smad5, Smad8. Optionally, the BMP inhibitor may be an indirect inhibitor of BMP. For example, the BMP inhibitor could be a compound or agent which induces expression of components required for gene knockdown or knockout of BMP pathway component. Examples of such a system may be DNA or RNA editing inducible programmable nucleases, notably the CRISPR/Cas9 system, small interfering RNAs, epigenetic editing systems.

The cells prepared according to the method of the invention are unique. A second aspect of the present invention, therefore, provides a composition comprising amniotic-like epithelial cells prepared according to the method as described herein. The composition can be a pharmaceutical preparation. The composition may include a scaffold such as a decellularized biological matrix or synthetic structure. It will be understood that the amniotic-like cells are applied to the matrix or scaffold after they have been prepared according to the methods of the invention, rather than being prepared in situ. The scaffold may be composed of any suitable material, and the scaffold chosen may depend upon the use to which the cells will be put. Suitable materials may or may not be biodegradable, and may include plastic polymers and metal. The composition may include a membrane, such as a biodegradable membrane, or a macroporous membrane made of polymers. The composition may include a gel such as a collagen gel, Matrigel™ or hydrogel. The composition may therefore comprise cells suspended in a gel. The composition can be a preparation for research purposes. The composition may be a cosmetic preparation.

The invention further extends to a composition prepared using the cells of the present invention. The cells are releasing compounds, factors and other chemicals that may be useful in the field of regenerative medicine. Therefore, the invention may usefully extend to a preparation derived from the cells of the invention, such as conditioned media, fractionated material from the media, extract of the cells, a homogenised preparation of cells, and extracellular extracts. As such, this aspect of the invention need not comprise live cells. Such may be useful where there are restrictions on the use of live cells for therapeutic uses, or if the use of live cells is undesirable.

The composition and/or cells of the present invention may be put to a variety of uses in relation to regenerative medicine and the like, in any human or animal subjects. The uses described herein are equally applicable to therapy in humans and veterinary medicine in animals. The compositions and/or cells of the present invention may be used in therapy. The compositions and/or cells of the present invention may be used in a method of treatment of the human or animal body in need of such treatment. The treatment may be any of those disclosed below.

The compositions and/or cells of the present invention may also have uses in cosmetic applications, such as in cosmetic surgery, in topical preparations such as creams. The compositions and/or cells of the present application may be used in methods of ameliorating or improving the appearance of wrinkles, fine lines, creases, crow’s feet, sagging skin, age spots and/or blemishes.

The composition and/or cells of the present invention may be used for wound healing and/or tissue repair, optionally skin repair or repair of muscle or connective tissue damage, such as a hernia or pelvic floor repair.

The composition and/or cells of the present invention may also be used for the treatment of ocular conditions or for ocular surface repair.

The composition and/or cells of the present invention may also be used for the treatment of burns, ulcers or surgical wounds.

The composition and/or cells of the present invention may also be used for treating diabetes or liver disease.

The composition and/or cells of the present invention may also be used for the treatment of congenital conditions, optionally epidermolysis bullosa.

The composition and/or cells of the present invention may also be used for the treatment of skin necrosis, optionally Stevens Johnson syndrome.

The composition and/or cells of the present invention may also be used for the treatment of urological and/or gynaecological conditions.

The composition and/or cells of the present invention may also be used as an anti-inflammatory.

Thus, there are a multitude of uses to which the cells prepared according to the methods of the present invention, as described herein, can be put. Most of these uses are in regenerative medicine.

A third aspect of the present invention provides amniotic epithelium prepared with cells differentiated according to the method described herein. This amniotic epithelium may be used therapeutically as described herein. The cells may also or alternatively be for use as a research tool. The amniotic epithelium may be supported on a scaffold such as a decellularized biological matrix or synthetic structure. The amniotic epithelium may be supported on a membrane, such as a polymer membrane. The amniotic epithelium may be suspended within a gel, such as a hydrogel.

A fourth aspect of the present invention provides a membrane prepared with cells differentiated according to the method described herein. The membrane may be used therapeutically as described herein. The membrane may additionally or alternatively be for use as a research tool. The membrane may be supported on a scaffold such as a decellularized biological matrix or synthetic structure.

A fifth aspect of the present invention provides a three dimensional (3D) structure, such as a hollow sphere or hollow spheroid, prepared with cells differentiated according to the method described herein. The 3D structure may be for use as a research tool.

A sixth aspect of the present invention provides a method of treatment of the human or animal body using the cells, compositions, epithelium or membranes as described herein. The method of treatment may include any therapeutic use of the amniotic-like epithelial cells, including wound healing or tissue repair, optionally skin repair.

A seventh aspect of the present invention is a method of preparing amniotic-like cells in suspension. The amniotic-like cells prepared in suspension according to the method described herein may form or provide a membrane or a three dimensional (3D) structure, such as a hollow sphere or hollow spheroid. Such a suspension-based method is suitable for commercial scale-up.

### DESCRIPTION OF THE FIGURES

FIGS. 1 (A to I). Characterisation of hALEC (human Amniotic Like Epithelial Cells): Human pluripotent stem cells (hPSC) (HNES1 line) after 3 days of capacitation in the presence of XAV939, followed by 5 days of differentiation in AP-containing media.

FIG. 1(A) is bright field microscopy, two focal planes of the same field of view. FIG. 1(B) shows diagnostic markers of pluripotency (POU5F1 and NANOG) and amnion (CDX2, HAND1, GATA2 and GATA3) during progression of amniotic lineage in ex vivo cultured human pre-gastrulation embryos (EPI is epiblast, EPI.AME is an intermediate stage between epiblast and amnion, AME is amnion); by single-cell RNAseq (Xiang et al. (2020) Nature, 577: 537-542). FIG. 1(C) shows the same markers during in vitro differentiation of hPSC to hALEC, assayed by qRT-PCR. FIG. 1(D) is a bright field microscopy of hALEC differentiated in suspension and FIG. 1(E) shows qRT-PCR for diagnostic markers during differentiation in suspension as compared to monolayer induction. FIGS. 1 (F and G) show immunostaining for markers GATA3, E-cadherin, CDX2, POU5F1, and fluorescently labelled Phalloidin is applied for counterstaining; and FIG. 1(H) depicts flow cytometry of hALEC, obtained from HNES1 capacitated for 5 days in XAV939 and then treated by AP for 4 days. FIG. 1(I) shows time-lapse imaging of hALEC self-assembly to epithelial bubbles.

FIGS. 2 (A to D). Comparison of hALEC to amnion cells of human and macaque embryos: Transcriptome of hALEC derived from HNES1 cells after 3-5 days of capacitation in the presence of XAV939, followed by differentiation in AP-containing media, was characterised by bulk and single-cell RNA sequencing. hALEC expression profile was compared to the cells of ex vivo cultured human pre-gastrulation embryos (Xiang et al. Nature 2020) and macaque gastrulating embryos (Ma et al. (2019) Science 366(6467): eaax7890, doi: 10.1126/science.aax7890). FIG. 2(A) shows average expression of pluripotent epiblast, early amnion and late amnion markers of embryos, during in vitro differentiation to hALEC. FIG. 2(B) shows clustering analysis of single cells in hALEC population, amnion-like cells are highlighted in black. FIG. 2(C) is analysis of fractions of identity (Gong et al (2013) Bioinformatics 29: 1083-1085) of embryonic cell populations in hALEC. FIG. 2(D) shows PCA of human and macaque embryo single cells, and undifferentiated cells and amnion-like cells in vitro. Respective lineages and cell types are highlighted in black. Abbreviations: hsAME.E – early amnion from human embryos; hsPostEPI – post-implantation epiblast from human embryos; hsTE –trophectoderm from human embryos; hsSTB-syncytiotrophoblast from human embryos; cyAME.L–late amnion from macaque (cynomolgus monkey) embryos.

FIGS. 3 (A to I). Signalling requirement for hALEC differentiation: FIG. 3(A) depicts experimental outline; naïve hPSC were capacitated in different conditions for 3 days (2uM XAV939 in N2B27 basal medium (“XAV939”), N2B27 basal medium only (“N2B27”), 1 uM A8301 in N2B27 basal medium (“A8301”), or medium E8 for culturing primed hPSCs (“E8”)), and then differentiated to hALEC in AP media. FIG. 3(B) is whole-well view of cells after hALEC induction stained with fluorescently labelled phalloidin. FIG. 3(C) shows qRT-PCR for diagnostic markers in cells before and after hALEC induction in 2 independent experiments. FIG. 3(D) shows brightfield images of hPSC (cR-H9-EOS line) that were capacitated for 3 days in N2B27 and then transferred to basal media either: without inhibitors (“None”), or with A8301 (“A”), or with PD03 (“P”), or with their combination (“AP”); or with both inhibitors and LDN193189 (“DAP”). FIG. 3(E) shows qRT-PCR results for characteristic markers in 2 independent experiments. FIGS. 3(F) and (G) show images of cells stained with fluorescently labelled phalloidin and qRT-PCR results, respectively, of hALEC differentiated in the presence of alternative MAPK inhibitors (1 uM PD0325901, or 5 nM, 10 nM or 30 nM Trametinib). FIGS. 3(H) and (I) show images of cells stained with fluorescently labelled phalloidin and qRT-PCR results, respectively, for hALEC differentiated in the presence of alternative TGFb pathway inhibitors (1 uM A8301; 10 or 20 uM SB431542; 1 uM or 5 uM LY2109761; 5 uM or 10uM or 20 uM LY364947).

FIGS. 4 (A to G). A competence window for hALEC differentiation during the formative transition: FIG. 4(A) depicts the experimental outline. hPSC (HNES1 line) were capacitated in XAV939 and analysed for their ability to form hALEC each day of capacitation, by treatment with AP of DAP for 4 days. FIG. 4(B) shows stitched images showing whole wells of a 24-well plate, and FIG. 4(C) shows individual fields of view. FIG. 4(D) is a qRT-PCR for markers in hALEC differentiated using AP medium after various length of capacitation. FIG. 4(E) depicts the immunofluorescence for markers (OCT4, CDX2 and E-cadherin) during the time course of hALEC differentiation using naïve and partially capacitated HNES1. FIG. 4(F) shows images of hALEC obtained from hPSCs capacitated for 8 days and then differentiated in AP medium in the presence of various BMP inhibitors (“LDN”, LDN193189; “Dorso”, Dorsomorphin; or K02288). FIG. 4(G) is the flow cytometry analysis.

### DETAILED DESCRIPTION OF THE INVENTION

The present invention relates to a method for differentiating pluripotent stem cells into amniotic-like epithelial cells, said method comprising culturing said cells with an inhibitor of the MAPK pathway and an inhibitor of the TGF pathway. This culturing may be described as ex vivo or in vitro and not in the human or animal body.

“Differentiation”, also known as “cellular differentiation”, involves a cell changing into another cell type; usually, but not always to a more specialised cell type. Differentiation occurs multiple times during the development of a multicellular organism. This process also carries on after the development of said organism, with focus on stem cells dividing to create fully differentiated daughter cells during tissue repair and during normal cell turnover. A cell’s size, shape, membrane potential, metabolic activity and responsiveness to signals can all change dramatically during differentiation due to highly controlled modifications in gene expression.

“Cell culturing” involves the removal of cells from an animal or plant which will then grow in favourable controlled conditions outside their natural environment, or “ex vivo”. The cell culture can then be used for in vitro assays. The cell culture can also be used to produce biological compounds such as antibodies or recombinant proteins. The conditions under which particular cells are cultured are important, particularly in relation to stem cell technologies. Culturing conditions vary for each cell type, but generally include the use of a suitable vessel with a medium that supplies the essential nutrients, growth factors, hormones, and gases, and regulates the physio-chemical environment. Most cells require a surface or an artificial substrate or a layer of feeder cells providing extracellular matrix and soluble factors (adherent or monolayer culture) whereas others can be grown free floating in culture medium (suspension culture).

Human amnion consists of AECs on a basement collagenous membrane, an acellular compact layer, a fibroblast layer, and a highly hygroscopic spongy layer. Amniotic epithelial cells (AEC) are usually extracted from the lining of the inner membrane of the placenta, using enzymatic digestion of the amnion membrane after it is separated from the underlying chorion. During development, the AEC are formed from epiblasts between day 7 and 9 after fertilization. AEC form squamous epithelium and express epithelial marker E-cadherin. AEC in human cultured pre-gastrulation embryos express the markers GATA2, GATA3, TFAP2A, TFAP2C, CDX2 and lack embryonic pluripotency markers NANOG, SOX2 and POU5F1 (Xiang et al (2020) Nature, 577: 537-542). AEC in term placentas express a specific combination of major histocompatibility complex antigens, including classical HLA-1a and nonclassical HLA-1b (HLA-E and placental-specific HLA-G) (Hammer et al (1997) Am J Reprod Immunol, 37(2): 161-171; Houlihan et al (1995) J Immunol, 154(11): 5665-5674). HLA-G is known to provide immunosuppressive properties to placenta (Le Bouteiller et al (1999) Hum Reprod Update, 5(3): 223-233).

The present invention relates to AECs which are derived in vitro/ex vivo from pluripotent stem cells. It is conventional, where cells have been derived effectively artificially, that the term “like” is applied to such cells. Thus, the cells of the present invention are “amniotic-like” epithelial cells. The AECs of the present invention are considered to be similar to those isolated from nature.

The cells of the present invention are amniotic-like epithelial cells generated from pluripotent stem cells. As such, the cells may have one or more of the following characteristics:
the cells are flat squamous epithelial cells;
the cells form a continuous layer of cells (an epithelium);and/or
the cells express one or more marker associated with amniotic epithelial cells such as E-cadherin (CDH1), CDX2, HAND1, TFAP2C, TFAP2A, GATA2, GATA3.

“Human amnion-like epithelial cells” (hALEC) are epithelial cells expressing amniotic epithelial cell markers generated from human pluripotent stem cells using culturing according to the method of the invention.

In general, the present invention relates to amniotic-like epithelial cells that form a continuous layer of cells, wherein said continuous layer of cells forms a membrane or a 3D structure. These cells can be human cells. These human amnion-like epithelial cells (hALECs) can have epithelial morphology and form large (up to 2 mm) hollow cysts in culture, reminiscent of amnion structure in the embryo. This is a 3D structure formed by the cells. The cells express genes characteristic of amnion cells, such as E-cadherin (CDH1), CDX2, HAND1, TFAP2C, TFAP2A, GATA2, GATA3. The inventors’ new approach therefore provides an expandable, standardised and potentially unlimited source of much sought-after proliferative human amniotic epithelial cells, all of which is an advantage over amnion cells from term placenta.

The amniotic-like cells of the present invention can form a continuous layer of cells. This continuous layer may be a layer of single cells. The layer is continuous, unbroken or whole layer of cells. Thus, the layer is composed of cells that are packed together. Epithelia are continuous sheets or layers of tightly linked cells that constitute the surfaces (such as the epidermis and corneal epithelium) and linings (such as the digestive, respiratory, and uro-genital epithelia) of the body. Thus, the amnion-like cells of the present invention may alternatively be described as being able to form an epithelial layer.

In nature, amniotic epithelial cells form part of the amniotic membrane. The epithelium cells, basement membrane and a stromal layer are the three major components of the amniotic membrane. The amniotic-like epithelial cells of the present invention may therefore require a biological matrix such as a decellularized matrix or a synthetic scaffold for use in certain embodiments. The cells may be applied after preparation according to the methods of the present invention. Decellularized or synthetic extracellular matrix (ECM) has emerged as a promising tool in the fields of tissue engineering or regenerative medicine. ECM provides a native cellular environment that combines its unique composition and architecture. It can be widely obtained from native organs of different species after being decellularized and provides necessary cues to cells homing. Biological scaffolds derived from extracellular matrix (ECM) have been widely utilised in regenerative medicine. These structures can be also created from synthetic components. Alternatively, a membrane such as a biodegradable membrane may be used to provide a support. Other options include using the cells in a gel, such as collagen or hydrogel.

The method of the present invention relies upon the culturing of pluripotent stem cells. These pluripotent stem cells may be any suitable pluripotent stem cell from any source. Pluripotent stem cells have the ability to undergo self-renewal and to give rise to all cells of the tissues of the body.

The pluripotent stem cells for use in the present invention may be any suitable source of cells, including embryonic stem (ES) cells, cells from parthenotes, embryonic stem (ES) cell lines, and induced pluripotent stem (iPS) cells. The cells may be human or animal. The pluripotent stem cells are preferably obtained without destruction of an embryo. It is possible to remove a single blastomere without embryo destruction. Induced pluripotent stem cells involve the reprogramming of somatic cells such as skin fibroblasts or blood cells using a variety of techniques, both genetic and chemical. The advantage of using somatic cells is that it enables autologous cells to be prepared, which will reduce the risk of rejection of the cells once transferred. However, due to the potential low expression levels of human leukocyte antigens (including HLA-A, HLA-B, and HLA-C and HLA-DR), which are key antigens involved in recipient rejection, allogenic preparations of cells from donor cells are also contemplated herein.

Pluripotency is defined as the ability of single cells to produce all lineages of the embryo. Pluripotency exists from emergence of the epiblast in pre-implantation blastocyst until lineage specification during gastrulation. This period lasts from ~4 days in rodents including mouse, to 8-10 days or longer in primates, including humans, and in many other mammals. Over this time, pluripotent epiblast cells change their properties from the initial naïve character to a primed state that is competent for differentiation. Both naïve and primed are states of pluripotency, but exhibit slightly different characteristics. The naïve state represents the cellular state of the preimplantation blastocyst inner cell mass, while the primed state is representative of the post-implantation epiblast cells. These two cell types exhibit clearly distinct developmental potential, as evidenced by the fact that naïve cells are able to contribute to blastocyst chimeras, while primed cells cannot. Those in the art consider that there may be a continuum of intermediate states between naïve and primed states in vivo, and thus a spectrum of cell types exist between these two extremes.

Naïve and primed states can be classified on the basis of multiple characteristics that each state can retain in vitro. Different combinations of exogenous factors confer distinct characteristics to pluripotent stem cells in vitro. As a result, cells acquire a distinct set of naïve and primed properties. It is possible that cells beyond the primed state are still pluripotent, as used herein “primed” encompasses cells beyond the point of primed.

Molecular criteria for defining the naïve human pluripotent state are described in Theunissen et al (2016) Cell Stem Cell, 19: 502-515, Oct. 6, 2016, herein incorporated by reference.

Naïve cells can be generated by resetting conventional primed stem cells, by somatic cell reprogramming, or by derivation directly from dissociated human inner cell mass (ICM) cells. They exhibit transcriptome correlation with the preimplantation epiblast and show protein expression of naïve epiblast-specific transcription factors such as KLF4, KLF17 and TFCP2L1.

Naïve cells are proposed to gain competence for lineage induction through a process of capacitation. The present inventor has previously established that the human naïve pluripotent stem cells lack competence to respond productively to inductive cues for lineage specification (Rostovskaya et al (2019) Development, 146(7): dev172916, herein incorporated by reference). Naïve hPSCs can be capacitated for somatic lineage induction.

The pluripotent stem cells as used in the method of the present invention may be any one or more of:
a. naïve pluripotent stem cells;
b. naïve pluripotent stem cells cultured under capacitating conditions;
c. primed pluripotent stem cells cultured under conditions reverting them to naïve pluripotent stem cells; and/or
d. pluripotent stem cells representing intermediate states between the naïve and the primed pluripotent states.

The cells of section (d) optionally include, but are not limited to, formative cells, cells that correspond to intermediates of the formative transition.

The pluripotent stem cell used in the method of the invention is preferably any pluripotent stem cell that is not in the primed state. The present inventors consider that it is possible that these cells are too far advanced down the capacitation pathway to enable the amnion-like epithelial cells to develop consistently and robustly. During embryogenesis, the inventors hypothesize than amnion-like epithelial cells are generated in advance of the primed state being achieved. However, it is possible to revert primed pluripotent stem cells towards the naïve state, and these reverted or partially reverted cells may be used in the method of the invention.

A “naïve pluripotent stem cell” is a pluripotent stem cell that can undergo differentiation into any of the three germ layers. These cells have the ability to generate chimeras in vivo due to their pluripotency. Naïve pluripotent stem cells do not respond to lineage induction or differentiation cues. Markers of naïve pluripotent stem cells include, but are not limited to, KLF4, TFCP2L1, DNMT3L, FGF4, KLF17, DPPA3 and DPPA5. Naïve pluripotent stem cells also express general pluripotency markers such as POU5F1, NANOG and SOX2. In addition, naïve pluripotent stem cells have low DNA CpG methylation levels (about 20-30%), in contrast to primed pluripotent stem cells and somatic cells (80-90% methylated CpG).

A “primed pluripotent stem cell” is a capacitated naïve pluripotent stem cell. It is possible to obtain these cells directly from human embryos, or alternatively they can be obtained via cell reprogramming. Such cells can be reliably induced to undergo productive differentiation into endodermal, mesodermal and neuronal cell types. These cells may exhibit dependence on exogenous FGF and activin/FGF for continued expansion. Primed pluripotent stem cells may express post-implantation markers, such as TCF7L1, TCF15, FGF2, SOX11, DUSP6, ZIC2 and HES1, in addition to general markers of pluripotency such as POU5F1, SOX2 and NANOG.

In vitro capacitation for multi-lineage differentiation may occur without exogenous growth factor stimulation and, under the specific conditions examined here, is facilitated by inhibition of Wnt signalling. Following capacitation, these cells can be induced to undergo productive differentiation into endodermal, mesodermal and neuronal cell types. The capacitation process of the cells may take up to about 10 days.

Pluripotent stem cells acquire the full spectrum of properties of primed cells after they have been capacitated for at least 10 days and then transferred to primed cell media with conditions suited for priming cells, herein referred to as primed cell media or primed cell conditions. The primed cell conditions, suitable for expanding such cells, may contain FGF2 and activin A, or alternative molecules activating the same signalling pathways, for further passaging. The global gene expression profile of the capacitated naïve cells becomes most similar to primed pluripotent stem cells after 10 days of capacitation and an additional 10 days of growth in primed cell conditions (Rostovskaya et al (2019)).

During embryogenesis, naïve cells go through a process of formative transition in order to reach the primed state, the late epiblast stage of development. The inventors consider that culturing naïve cells under capacitating conditions allows them to track the process of formative transition. In the human embryo, pluripotency is a characteristic of epiblast cells from the early pre-implantation stage until lineage specification during gastrulation, lasting for at least 10 days. During this window, the epiblast cells progress through several distinct developmental phases and therefore, pluripotency is a generic property of cells with different identities. As such, two extreme states of pluripotency have been defined: naïve cells correspond to the early pre-implantation epiblast and primed cells are reminiscent of the pre-gastrulation stage. By using specific culture conditions, human pluripotent stem cells (hPSC) resembling these distinct states can be isolated and propagated in vitro retaining their properties. The inventors have previously established previously a culture system for the controlled transition of naïve cells toward the primed state in vitro, in a process termed formative transition or capacitation (Rostovskaya et al (2019) Development, 146(7): dev172916). Importantly, gene expression analysis confirmed that the formative transition in vitro recapitulates the transcriptional changes that occur during the in utero development of primate embryos (Rostovskaya et al (2019)).

During capacitation, the cells pass through a developmental continuum. The expression of various markers start associated with the naïve state start to decline, and the expression of markers associated with post-implantation start to increase. Capacitation is a process that is continuous and seamless, with the cells leaving the naïve state and moving towards the primed state. It is during this process of capacitance that the inventors have developed a process to stably produce amnion-like cells. It is thought that the pluripotent stem cells are competent to produce amnion-like cells during the progression from pre-implantation (naïve) state to post-implantation (primed) state, reflecting the properties of the peri-implantation epiblast.

Human naïve and primed pluripotent cells have distinct signalling requirements for sustained self-renewal in vitro (Takashima et al (2014) Cell, 158(6): 1254-1269; Theunissen et al (2014) Cell Stem Cell, 15(4): 471-487). The maintenance of naïve hPSC requires the inhibition of the mitogen-activated protein kinase (MAPK) pathway, whereas the propagation of primed hPSC depends upon the activity of this pathway (Vallier et al (2005) JCS, 118: 4495-4509). Furthermore, active TGFb/Activin/Nodal signalling facilitates the stable maintenance of naïve hPSC (unpublished data) and is strictly necessary for primed hPSC to self-renew (Vallier et al (2009) Development, 136(8): 1339-1349). Since the discovery of the system for formative transition, the inventors have sought to identify the stage during the progression from naïve to primed state when hPSC switch their signalling requirements for self-renewal. Excitingly, and unexpectedly, they have found that upon the simultaneous inhibition of the TGFb/Activin/Nodal and MAPK pathways, hPSC at several stages along the capacitation process form epithelial cells that rapidly self-assemble into adherent, 3D hollow spherical structures. The character of these cells were analysed and it was unexpectedly revealed that they possessed amnion epithelium identity.

It may be preferred that the pluripotent stem cells have exited from the naïve state before the cells are differentiated into amniotic-like epithelial cells. Pluripotent stem cells may be exited from the naïve stage by culturing under capacitation conditions. However, as described below, naïve cells can be used to generate amniotic-like cells, but they may require additional time compared to cells cultured under capacitating conditions.

It may be preferred that the pluripotent stem cells have not reached the primed state before the cells are differentiated into amniotic-like stem cells. Primed stem cells may be reverted to an earlier state using various previous methods (Theunissen et al (2014) Cell Stem Cell, 15(4): 471-487; Takashima et al (2014) Cell, 158(6): 1254-1269; Guo et al (2017) Development, 144(15): 2748-2763).

There may be a window during the progression from naïve to primed stem cells during which the cells are optimally positioned to differentiate into amnion-like cells.

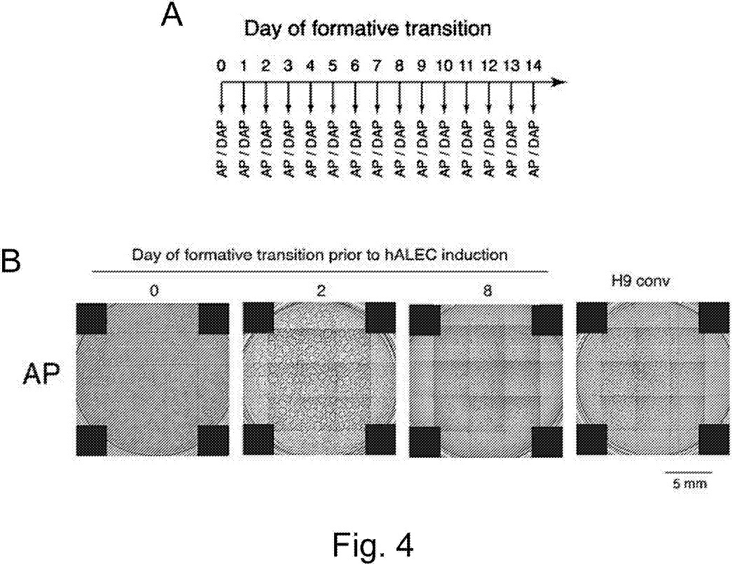

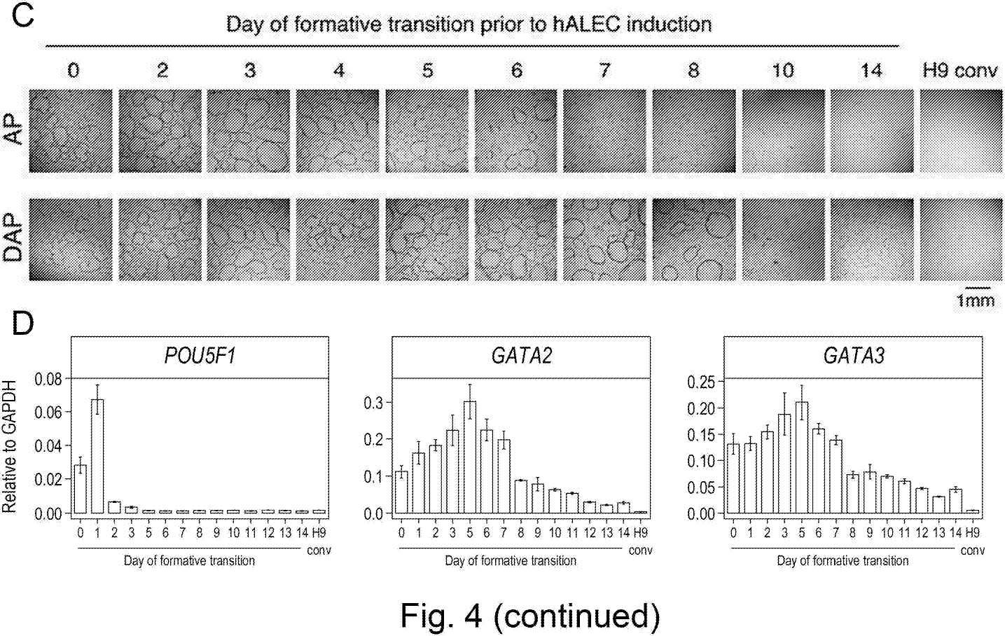

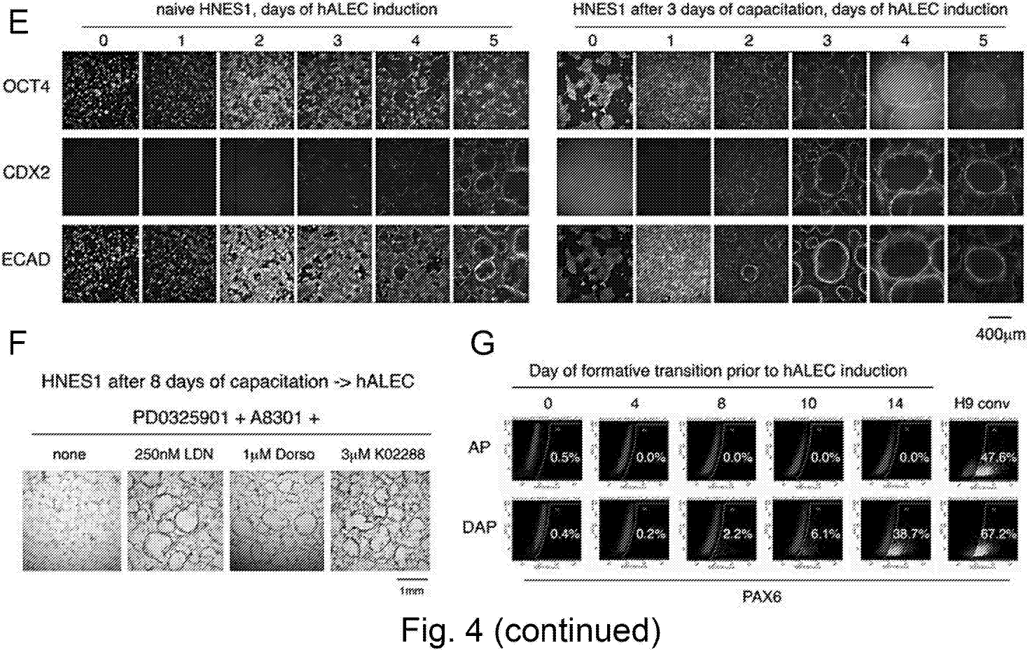

To identify a possible window during the progression from naïve to primed pluripotency where hPSC have the competence to form amnion-like cells, systematic testing of the ability of hPSC at different stages of capacitation to respond to hALEC-inducing cues (FIG. 4C) was carried out. Interestingly, naïve hPSC without prior capacitation (day 0) produced epithelial spheres, however, the emergence of these spheres was delayed by at least one day (FIG. 4E) and the efficiency of sphere formation was reduced. The observed delay in the response by naïve hPSC suggests that the exit from naïve pluripotency may be required prior to hALEC formation. Notably, if hALEC differentiation was induced at any time point of hPSC capacitation beyond day 0, the spheres that emerged did so simultaneously when comparing between these cell populations and the differentiation showed similar dynamics. It is important to note that hPSC strongly downregulate the pluripotency markers that define the naïve state, for example KLF4, after only 1 day of capacitation. hPSC only gain the transcriptional signature most similar to primed hPSC after 10 days of capacitation and further passaging in primed cell media (under the conditions described herein).

hPSC gain the transcriptional signature most similar to primed hPSC only by about day 10 of capacitation. Hence, the competence window to produce amniotic epithelium may be seen to encompass a period of formative transition that occurs after the exit from the naïve state and before the acquisition of the primed state.

The pluripotent stem cells may be cultured under capacitating conditions. These conditions may vary according to the conditions selected to maintain the cells in a naïve state. Capacitating conditions generally involve the withdrawal of self-renewal conditions. Self-renewal conditions may require the presence of growth factors, chemical inhibitors and other components promoting self-renewal. In the Examples, the following components are considered to aid self-renewal and are withdrawn: PD0325901, Go6983, XAV939 and LIF. However, those skilled in the art will appreciate that there are various protocols to culture naïve hPSC, so this combination of components can differ as appropriate. Conditions may include an absence of exogenous growth factor stimulation. Alternatively put, the cells are cultured in basal conditions. In some situations additional components may be added that do not interfere with capacitation, such as FGF2, activin A or TGFb inhibitors (example - A8301), or BMP inhibitors (example - LDN193189). Optionally, the cells can be contacted with an inhibitor of Wnt. Such conditions allow the cells to gain competence over about 7-10 days for efficient differentiation into neuroectoderm, definitive endoderm and mesoderm lineages.

As used herein, a pluripotent stem cell may be at any stage of capacitation, from naïve to primed, but is preferably between these two stages. Naïve cells are capable of differentiation, but their development is delayed by about 24 hours. Primed pluripotent stem cells are cells derived directly from embryos or by reprogramming of somatic cells using conditions that include FGF2 and activin A (or related growth factors activating the same pathways) and further expanded in these conditions, or by capacitation of naïve pluripotent stem cells for at least 10 days and then grown in media containing FGF2 and activin A for a further 10 days.

The pluripotent stem cell has, therefore, preferably exited the naïve state but not yet reached the primed state. Cultured according to the capacitating conditions disclosed here, this can be correlated with cells that have been cultured under such conditions and not transferred to the conditions for maintenance of the primed pluripotent stem cells.

The skilled person would be able to use any suitable method of capacitating the pluripotent stem cells, and different methods may take different number of days before the cells reach the point of being capacitated. The final step of acquisition of primed phenotype occurs after the capacitated cells have been transferred to primed media. If the capacitating conditions as described in the Examples or similar conditions used for the capacitation, then formative transition requires (about) at least 10 days, prior to transfer to primed media. The period of culture in capacitation conditions can be extended beyond 10 days, in this case the capacitated cells can’t be maintained indefinitely and do not acquire all properties of primed cells. In the capacitation conditions similar described here, pluripotent stem cells can be cultured for up to 18 days before they begin to spontaneously to differentiate. Thus, the pluripotent stem cells may have therefore been cultured under capacitating conditions for any one of 1, 2, 3, 4, 5, 6, 7, 8, 9, 10, 11, 12, 13, 14, 15, 16, 17, 18 days. Optimally, the cells are cultured under capacitating conditions for 2 to 12 days, 2 to 10 days, 2 to 6 days, optionally 2 to 5 days.

Use of a BMP inhibitor may allow the window to be extended up until shortly before the primed state is acquired. Thus, when the method of the invention involves the culturing of cells with a BMP inhibitor, this permits the pluripotent stem cells to be previously cultured in capacitating conditions for longer, extending the window of maximal efficiency of differentiation. Optimally, the cells may be subjected to capacitating conditions for 2 to 9 days, suitably 2 to 8 days, optionally 2 to 7 days.

Such windows in the formative transition are demonstrated in FIGS. 4A to 4G.

Those skilled in the art will appreciate that using different capacitating conditions will result in different timescales for capacitance. Therefore, the invention preferably uses a pluripotent stem cell that can be defined as one cell type on the developmental continuum between the naïve and primed states. It is preferred that the pluripotent stem cell is not in the primed state, but can be a primed stem cell that has reverted to an earlier cell type in the developmental continuum.

In order to differentiate the pluripotent cells into amnion-like cells, the pluripotent cells may be cultured with various inhibitors in order to direct the cells to an amnion-like state.

The method of the present invention may comprise the use of a MAPK pathway inhibitor. This MAPK pathway inhibitor can be a chemical inhibitor, neutralising antibody, aptamer, ligand trap, antisense nucleotide, protein inhibitor, and engineered peptide, targeting any one of the pathway components selected from the list comprising: receptor tyrosine kinases, Ras, Src, Raf, MEK½, p38 MAP kinases, ERK½; or activators or agonists of AKT and PI3K. Optionally, the MAPK pathway inhibitor may be an indirect inhibitor of the MAPK pathway. For example, the MAPK inhibitor could be a compound or agent which induces expression of components required for gene knockdown or knockout of a MAPK pathway component. Examples of such a system may be DNA or RNA editing inducible programmable nucleases, notably the CRISPR/Cas9 system, small interfering RNAs, epigenetic editing systems.

In an embodiment, the MAPK pathway inhibitor may inhibit any one or more of the direct components of the MAPK pathway, including RAS, RAF, MEK½ and/or ERK½ (MAPK). Inhibition of MEK1/MEK2 may be particularly desirable.

The mitogen-activated protein (MAP) kinases are ubiquitous intracellular signalling proteins that respond to a variety of extracellular signals and regulate most cellular functions including proliferation, apoptosis, migration, differentiation, and secretion. The four major MAP kinase family members, which include the ERK½, JNK, p38, and ERK5 proteins, coordinate cellular responses by phosphorylating and regulating the activity of dozens of substrate proteins involved in transcription, translation, and changes in cellular architecture. Many inhibitors of the MAPK pathways are under investigation, notably as they are being developed as cancer therapeutics.

Exemplary chemical inhibitors of this pathway include:

Receptor tyrosine kinase inhibitors targeting EGFR : Gefitinib (Iressa®), targeting VEGFR: Erlotinib (Tarceva®), Lapatinib (Tykerb®), targeting PDGFR : Sunitinib (Sutent®), Sorafenib (Nexavar®), targeting FGFR: PD173074, SU5402.

Non-receptor and receptor tyrosine kinase inhibitors targeting Bcr-Abl: Nilotinib (Tasigna®), targeting Bcr-Abl, c-Src: Dasatinib (Sprycel®), targeting Bcr-Abl, c-SCT, c-Kit, PDGFR: Imatinib (Gleevec®). G-protein inhibitors, targeting Ras: Tipifarnib (Zarnestra™).

MAPKKK inhibitors, targeting Raf: Sorafenib (Nexavar®), Sorafenib Tosylate, Dabrafenib, Regorafenib, RAF265, PLX-4720, LY3009120, RAF709, GDC-0879,

MAPKK inhibitors targeting MEK½: PD0325901, GSK1120212, PD98059, U0126, PD184352, and AZD6244; targeting MEK5: BIX02188, BIX02189.

MAPK inhibitors targeting p38: SB203580, SB202190, BIRB-796, Doramapimod

In the Examples, PD0325901 (MEK½ inhibitor) and Trametinib (GSK112021, MEK½ inhibitor) are used.

Antisense nucleotides are available that target components of the MAPK pathway. Further, it is possible to obtain blocking peptides and neutralising antibodies to MAPK pathway components.

The method of the present invention may comprise the use of a TGF pathway inhibitor. This TGF pathway inhibitor can be a chemical inhibitor, neutralising antibody, ligand trap, antisense nucleotide, protein inhibitor, or engineered peptide, targeting any one of the pathway components from the list comprising: ligands TGF beta, Activin, Nodal; TGF beta type I receptors TGFBR1, ACVR1, ACVRL1, ACVR1B, ACVR1C; TGF beta type II receptors TGFBR2, ACVR2A, ACVR2B; signal transducers Smad2, Smad3, Smad4; TGF ligand processing enzyme furin. Optionally, the TGF pathway inhibitor may be an indirect inhibitor of the TGF pathway. For example, the TGF inhibitor could be a compound or agent which induces expression of components required for gene knockdown or knockout of a TGF pathway component. Examples of such a system may be DNA or RNA editing inducible programmable nucleases, notably the CRISPR/Cas9 system, small interfering RNAs, epigenetic editing systems.

The inhibitor may be active against a TGF beta-receptor type I or TGF beta-receptor type II. Alternatively or additionally, the inhibitor may inhibit the activin A receptor (ACVR1C or ALK-7) and/or activin receptor type-1B (ACVR1B or ALK-4). Alternatively or additionally, the inhibitor is one which inhibits SMAD signalling but optionally does not inhibit BMP signalling.

The transforming growth factor beta (TGF) signalling pathway is involved in many cellular processes in both the adult organism and the developing embryo including cell growth, cell differentiation, apoptosis, cellular homeostasis and other cellular functions. TGFβ superfamily ligands bind to a type II receptor, which recruits and phosphorylates a type I receptor. The type I receptor then phosphorylates receptor-regulated SMADs (R-SMADs) which can now bind the coSMAD SMAD4. R-SMAD/coSMAD complexes accumulate in the nucleus where they act as transcription factors and participate in the regulation of target gene expression.

Exemplary chemical inhibitors of the TGF signalling pathway include:
Pan TGF-β inhibitors: 2G7, SR-2F, ID11, GC-1008.
TGF-β2 inhibitors: Metelimumab (CAT-192),
TGF-β⅔ inhibitors: Lerdelimumab (CAT-152)
TGFβRI & RII kinase inhibitors: LY-2109761
TGFβRI kinase inhibitors: LY-550410, LY-580276, LY-2157299, LY-573636, LY364947, SB-505124, SB-
431542, SD-208, Ki-26894, Sm16, NPC-30345, A-83-01, SX-007, IN-1130.

In the Examples, A-83-01 (TGFβ receptor type I kinase inhibitor), SB431542 (TGFβ receptor type I inhibitor), LY2109761 (dual TGFβ receptor type I and type II inhibitor) and LY364947 (Selective TGFβ receptor type I inhibitor) are used.

Exemplary antisense oligonucleotides of components of the TGF signalling pathway include:
AP-12009 targeting mRNA TGF-β2
AP-11014 targeting mRNA TGF-β1
NovaRx antisense targeting TGF-β1 & TGF-β2

Exemplary interacting peptide aptamers targeting Smads: Trx-xFoxH1b.

Preferably, the method of the invention comprises the use of a MAPK pathway inhibitor and a TGF pathway inhibitor. Optionally, the method of the invention may also comprise the use of a BMP inhibitor. The method of the invention therefore comprises culturing the pluripotent stem cells with a MAPK pathway inhibitor, a TGF pathway inhibitor and optionally a BMP inhibitor.

The method of the present invention may also comprise the use of a BMP inhibitor. This BMP inhibitor can be a chemical inhibitor, neutralising antibody, ligand trap, antisense nucleotide, protein inhibitor, engineered peptide targeting any one from the list comprising: ligands BMP2, BMP4, BMP7; BMP type I receptors BMPRIA, BMPRIB; BMP type II receptor BMPR2, Smad1, Smad5, Smad8. Bone morphogenetic proteins (BMP) are embryonic proteins that are part of the transforming growth factor (TGFβ) superfamily. Optionally, the BMP inhibitor may be an indirect inhibitor of BMP. For example, the BMP inhibitor could be a compound or agent which induces expression of components required for gene knockdown or knockout of BMP pathway component. Examples of such a system may be DNA or RNA editing inducible programmable nucleases, notably the CRISPR/Cas9 system, small interfering RNAs, epigenetic editing systems.

The inhibitor may target any one or more of: bone morphogenetic protein receptor type IA (BMPR1A or ALK3), activin A receptor type I (ACVR1 or ALK-2 (activin receptor-like kinase-2)), Bone morphogenetic protein receptor type-1B (CDw293, BMPR1B or ALK6), and/or serine/threonine-protein kinase receptor R3 (ACVRL1 or ALK1).

Exemplary inhibitors of BMP include:

K02288, DMH1, DMH2, LDN 193189 hydrochloride, dorsomorphin and analogues thereof, LDN 212854 trihydrochloride and Noggin.

Exemplified here are LDN193189 (ALK2, 3 and 6 inhibitor), dosomorphin (ALK2, 3 and 6 inhibitor) and KO2288 (ALK1, 2, 3 and 6 inhibitor).

Inhibition of a MAPK pathway component, TGF pathway component or BMP pathway component may be indirect, for example through inducible gene/DNA/RNA/epigenetic editing to knock-out or knock-down a suitable component, such as BMP. Inducible gene editing generally makes use of inducible promoters that are “switched on” in the presence or absence of a compound (such as a drug) and then allow the production of a component required for the gene or RNA editing. Such inducible promoters include the Tet-on/off system which requires doxycycline for induction, or lactose (Lac)/repressor (Lacl) system which requires isopropyl β—D—1-thiogalactopyranoside (IPTG), or ER/ERT2 system which requires tamoxifen.

Various methods are available for gene (DNA) or RNA editing that would allow for temporary or permanent knock-down or knockout of gene function. RNA editing is by nature a temporary way of knocking out gene expression. DNA or gene editing can be both temporary and permanent.

Various methods of gene, DNA or RNA editing exist. RNA editing can be achieved by using pre-existing ADAR (adenosine deaminases acting on RNA) enzymes in the cell, and providing a guide RNA. RNA editing may also be achieved with a modified CRISPR/Cas9 system described further below.

CRISPR gene editing uses a guide RNA to direct an enzyme called Cas9 to a complementary DNA strand, or RNA strand in the case of RNA editing. Many modifications of Cas9 are available to alter various properties, including removing its ability to cleave nucleic acid entirely. For example, modified CRISPR/Cas 9 systems have been designed that allow for different effects. CRISPRi (CRISPR interference) and CRISPRa (CRISPR activation) are two such modifications. CRISPRi silences genes at the transcriptional level, whilst CRISPRa can be utilised to upregulate gene expression. In the CRISPRi system, a catalytically dead Cas 9 (dCas9) is expressed, lacking endonuclease activity, with the guide RNA (gRNA). The gRNA is complementary to the gene of interest.

Gene editing can also be achieved using other systems such as zinc-finger nucleases, transcription activator-like effector nucleases (TALENs), and meganucleases. Such techniques rely on cellular DNA-repair mechanisms in order to effect the gene editing.

Aptamers may also be used as inhibitors of the various pathway components. The entities are oligonucleotide or peptide molecules that bind to a specific target molecule. Aptamers are usually created by selecting them from a large random sequence pool, but natural aptamers also exist.

Indirect inhibition of MAPK, TGFb or BMP pathway can be achieved by inducible protein degradation, to eliminate components of the respective pathways. Examples of inducible protein degradation systems include AID degron system and TRIM-away. AID degron inducible protein degradation system employs tagging protein of interest with a small peptide (AID) and expression of TIR1 protein in the same cell; adding plant hormone auxin causes degradation of the respective protein. TRIM-away system involves expression of TRIM21 protein in the cells, delivery of an antibody into the cell causes degradation of proteins that carry the epitope.

Thus, the inventors have established that the minimal conditions required to induce human amnion-like epithelial cell development is the use of a TGF pathway inhibitor with a MAPK pathway inhibitor. A BMP inhibitor may also be added to the induction culture, and may have the effect of changing the window in which the cells may be differentiated. In the Examples, it is shown that blocking BMP signalling does not interfere with hALEC differentiation (FIG. 4C). This, therefore, shows the discovery of a unique, BMP-independent, route to human amnion differentiation in vitro. The inhibition of the BMP pathway potentiated the inhibition of the MAPK and TGFb pathways, at least at the later stages of capacitation, and extended the window of competence.

The pluripotent stem cells may be cultured with a TGF pathway inhibitor with a MAPK pathway inhibitor, and optionally a BMP inhibitor under any suitable conditions, notably in an adherent culture or in suspension. Adherent cells are cells which must be attached to a surface to grow, and are commonly used in laboratory environments. However, to produce commercial scales of cells, the preference has been to use suspensions of cells. Thus, the pluripotent stem cells may be cultured in suspension using non-adhesive tissue culture plates or bioreactors. Using bioreactors permits large quantities of cells to be produced under cGMP (current Good Manufacturing Practices) conditions. Preferably, the culture conditions are serum-free. Preferably, the method involves dissociating the pluripotent stem cells, transferring to non-adhesive culture plates or culture bags, suitably at a seeding density of about 4×10⁵ cells/ml in a differentiation medium. Suitably, the differentiation medium may comprise ROCK inhibitor, suitably at a concentration of about 10 µM, optionally for about the first 24 hours of differentiation. The cells maybe cultured under appropriate conditions, such as a CO₂ incubator. Such a method is described in the Examples.

The cells as prepared herein may be further differentiated to other cell types if desirable, using appropriate conditions.

The inventors consider that these amniotic-like cells derived from pluripotent stem cells have been generated for the first time. Therefore, these cells developed in the lab are new. Cells derived by the method described herein form part of the present invention. These cells are amniotic-like, and are thus similar to the natural cells. The cells can be supplied in a substantially pure preparation, such that the cells present are at least 90%, at least 95%, 96%, 97%, 98% or 99% pure, such that cells of other types are not present.

The present invention also relates to a composition comprising amniotic-like epithelial cells prepared according to the method of the invention. Alternatively, the composition may comprise a preparation derived from the amniotic-like cells of the invention, including homogenised cells, cell extracts, cell culture medium and extracts thereof. These compositions may be a pharmaceutical preparation. The composition may include a scaffold. These compositions may be a cosmetic preparation.

The present invention further relates to the use of the cells or compositions disclosed here in therapy and/or methods of treatment of the human or animal body in need thereof. The cells may be autologous (derived from the patient) or allogenic (derived from a donor).

The present invention further relates to the use of the disclosed composition and/or cells in regenerative medicine. Examples of potential uses of the cells or composition include wound healing and/or tissue repair, optionally skin repair. The composition and/or cells can also be used for the treatment of ocular conditions or for ocular surface repair. Additionally, the composition and/or cells can be used for the treatment of burns, ulcers, surgical wounds, diabetes, and liver disease. The composition and/or cells can also be used for the treatment of congenital conditions, optionally epidermolysis bullosa, or skin necrosis, optionally Steven Johnson syndrome. Furthermore, the composition and/or cells can be used for the treatment of urological and/or gynaecological conditions. The composition and/or cells can also be used as an anti-inflammatory.

The term “pharmaceutical preparation” in the context of this invention means a composition comprising an active agent and comprising additionally one or more pharmaceutically acceptable carriers. These carriers may be a gel (such as a collagen gel or hydrogel), a membrane (such as a biodegradable membrane or thin polymer membrane) or a scaffold.

The term “cosmetic preparation” in the context of this invention means a composition comprising an active agent and comprising additionally one or more cosmetically acceptable carriers. Said cosmetic preparation may comprise one or more excipients or carriers suitable for carrying the cells of the invention to the application site, typically the skin, such as the face, or it may comprise a cosmetic formulation containing one or more further components with a cosmetic action. Typically, the cosmetic formulation or base wherein the complex of stem cells of the invention can be dispersed may comprise one or more excipients or substances commonly used for cosmetic applications and for the formulation of creams, for example glycerin, substances with a fatty base such as fatty acids and derivatives thereof, triglycerides, oils, emulsions, thickeners, liposomes, glycols, alcohols, preservatives, silicones, humectants, emollients, and also active principles or vitamins commonly used in the cosmetic field such as vitamin C, vitamin E and derivatives thereof, hyaluronic acid, sunscreens, fructose, peptides, ribonucleic acids and derivatives thereof.

The present invention may also relate to amniotic epithelium prepared with cells differentiated according to the method of the invention. The present invention may further relate to a membrane prepared with cells differentiated according to the method already disclosed. The present invention also relates to a three-dimensional structure, such as a hollow sphere or hollow spheroid, prepared with cells differentiated according to the method of the invention. The cells, membranes or structures disclosed in the present invention can also be used as a research tool.

The present invention may relate to a method of treatment comprising use of the cells, composition, membrane or epithelia as described herein. The method of treatment may be for wound healing or tissue repair, optionally skin. The method of treatment may be the treatment of ocular conditions or for ocular surface repair, the treatment of burns, ulcers, surgical wounds, diabetes, and liver disease. The method of treatment may be the treatment of congenital conditions, optionally epidermolysis bullosa, or skin necrosis, optionally Steven Johnson syndrome. The method of treatment may be the treatment of urological and/or gynaecological conditions. The method of treatment may be the treatment of inflammation.

The present invention may relate to a method of cosmetic treatment comprising use of the cells, composition, membrane or epithelia as described herein. The methods of treatment may ameliorate or improve the appearance of wrinkles, fine lines, crow’s feet, creases, sagging skin, age spots and blemishes.

All references to publications made herein are incorporated by reference for the purposes of US patent prosecution.

The invention as described herein is now exemplified by the following non-limiting examples:

### EXAMPLES

### 1. Human Pluripotent Stem Cells Can Produce Amnion-like Epithelial Cells

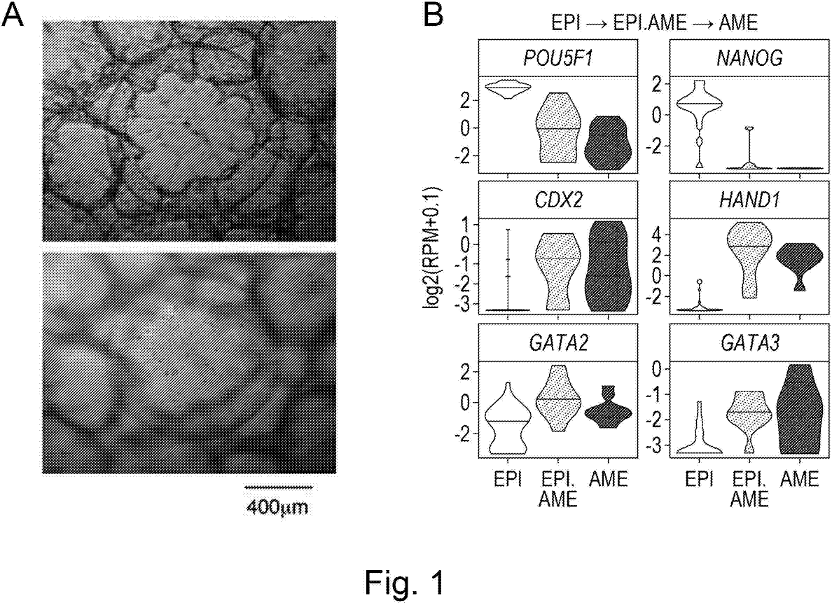

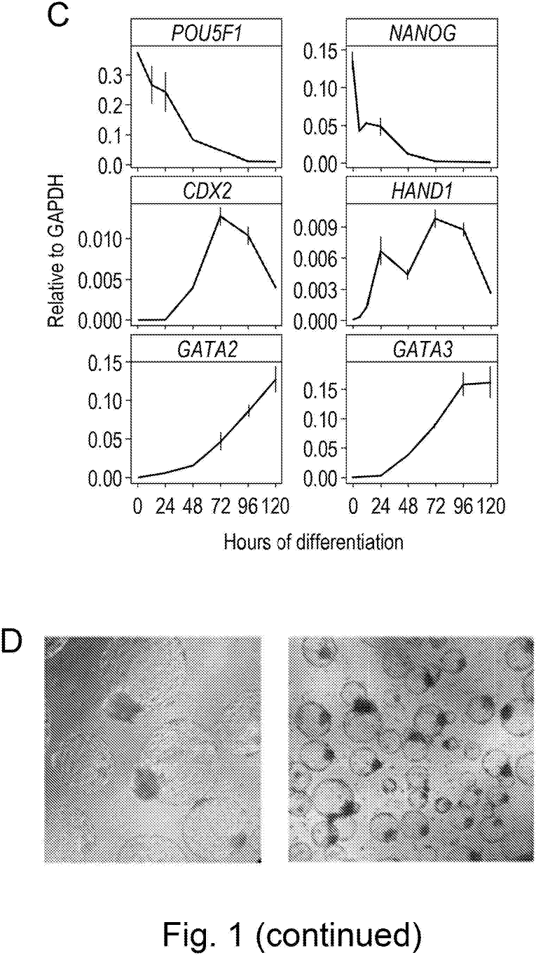

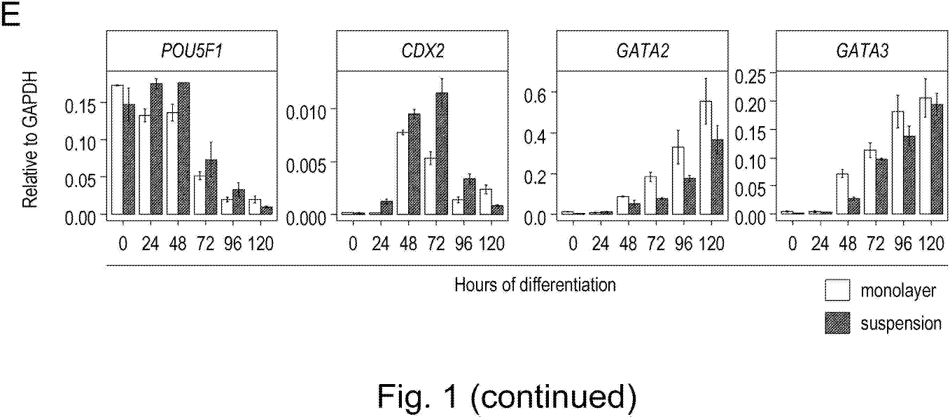

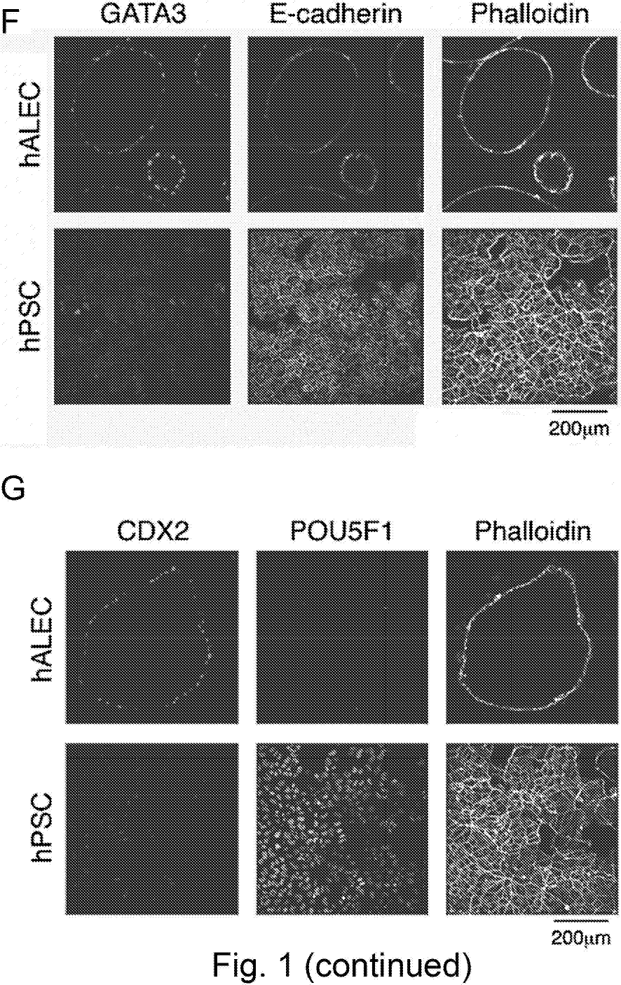

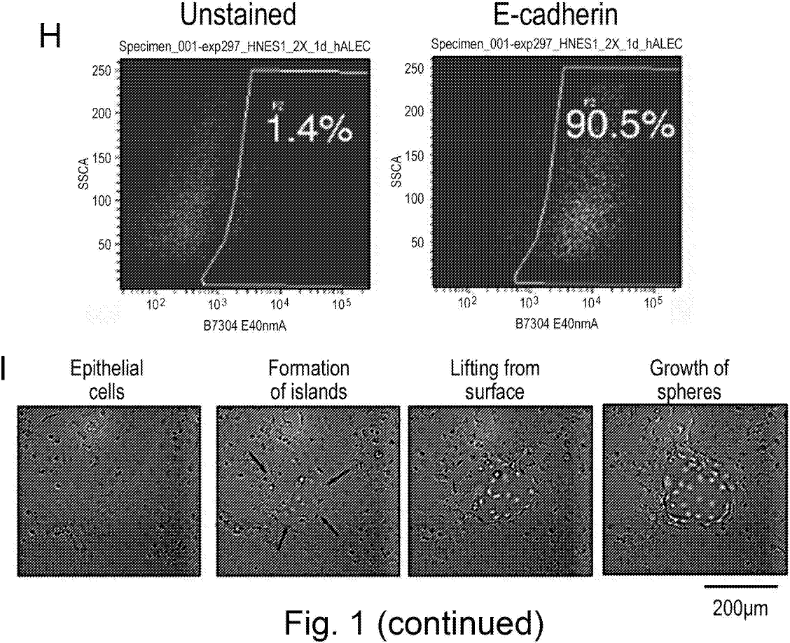

To initiate formative transition, naïve hPSC were treated for 3 days with the tankyrase inhibitor XAV939 that suppresses WNT signalling (Rostovskaya et al. Development 2019). These partially capacitated cells were then transferred to a medium containing two inhibitors termed “AP” consisting of: A8301, which blocks the activation of TGFb receptors (ALK-4, -5, -7); and PD0325901 (PD03 thereafter), which is an inhibitor of MAPK/ERK kinase (MEK). Inhibitor of Rho kinase (ROCK) was added during the first 24 hours of differentiation to improve cell viability, then could be omitted. After 5 days in AP-containing medium, the cells spontaneously formed numerous 3D structures with the appearance of hollow bubbles that grew out of the monolayer remaining attached to the culture dish (FIG. 1A). Formation of bubbles was observed when the cells were plated at a density allowing them to grow to monolayer, optimally 10⁵/cm² (however as low as 10⁴/cm² density resulted in spheres formation).

To investigate the identity of the epithelial bubbles generated by partially capacitated hPSC in response to AP, we characterised the expression of diagnostic mRNA and protein markers in these cultures. Because our previous work demonstrated that hPSC gain the capacity to differentiate into somatic lineages during the formative transition (Rostovskaya et al. Development 2019), we investigated the expression of somatic lineage markers, such as ectoderm (SOX1, PAX6), endoderm (SOX17, GATA4) and mesoderm (TBRA), but these genes were not detected in the AP-treated cells (results not shown), thus ruling out a possibility that they belong to embryonic germ layers. In primate embryos, including human, prior to differentiation to somatic lineages pluripotent epiblast cells form amniotic epithelial cells during implantation (Luckett Dev Dynam 1975; Enders et al. Am J Anat 1986; Nakamura et al. Nature 2016; The Virtual Human Embryo Atlas). This period of embryo development in utero corresponds closely to an intermediate stage of the formative transition of hPSC in vitro. Therefore, we tested for the presence of markers that are characteristic of amnion in primate and human development such as CDX2, HAND1, GATA2 and GATA3 (Shao et al. Nat Mater 2017; Shao et al. Nat Commun 2017; Xiang et al. Nature 2020) and for the loss of pluripotency markers POU5F1 and NANOG. We detected consistent dynamics of these genes expression in amniotic lineage of ex vivo cultured pre-gastrulation human embryos according to the published single cell RNAseq dataset (Xiang et al. Nature 2020) and during our differentiation in AP conditions (FIGS. 1B and C).

The spheres were also formed when partially capacitated cells were cultured in AP medium in suspension, in non-adhesive tissue culture plates (FIGS. 1D and 1E). Suspension-based differentiation of hPSC to hALEC was achieved as follows. hPSC were dissociated to single cells using TrypLE Express and counted. The cells were plated to non-adhesive tissue culture plates (such as Corning Costar Ultra-Low Attachment plates, Cat. CLS3471) at a seeding density of 4×10⁵/ml in differentiation medium with 10 µM ROCK inhibitor, and further cultured on a rocker platform in a CO₂ incubator. Differentiation medium was prepared as following: N2B27 basal medium, 1 µM PD0325901 and 1 µM A8301 (Cat. 2939, Tocris Bio-Techne). The medium was changed daily. ROCK inhibitor was required for the first 24 hours of differentiation to improve cell viability, then can be omitted.

In addition, the majority of cells expressed proteins that are present in amnion including GATA3 and CDX2 but not the pluripotent epiblast marker OCT4 (FIGS. 1F and G). The cell surface marker E-CADHERIN was detected in >90% of the cells, thereby confirming their epithelial identity (FIGS. 1F and H). These data demonstrate that hPSC that have undergone 3 days of the formative transition, generate epithelial cells expressing amniotic epithelial cell markers in response to AP treatment. Based on these properties, we have termed the cells “human amnion-like epithelial cells” (hALEC).

### 2. Tracking of hALEC Self-Assembly Into Epithelial Spheres

Time-lapse microscopy was used to investigate the morphological changes during hALEC differentiation (FIG. 1H). Within 24 hours after plating the partially capacitated hPSC into AP-containing media, the cells acquired clear epithelial cell morphology. Over the next 16 hours, these cells formed epithelial islands with distinct borders separating them from the surrounding cells. The islands then began to lift away from the surface. Most of the 3D structures, now resembling bubbles on a dish, emerged within a 6-hour window, typically beginning ~40 hours after the application of AP. The spherical structures grew by enlarging the size of the constituent cells, by engaging more epithelial cells from the 2D monolayer, and by fusion with other spheres (results not shown). After this time period, the spheres sometimes collapsed and reformed, however, the emergence of new spheres was rarely observed. The spheres grew rapidly and they typically reached their maximum size by 96-120 hours. Sphere diameter is variable and can reach 1-2 mm (FIGS. 1A, 1F, 1G, 3B, 3D, 3F, 3G, 4B, 4C, 4E, 4F). The exact timing of these events slightly depended on the starting cell density, however, overall, the process is remarkably consistent between experiments (currently >30 independent experiments using 4 hPSC lines).

### 3. Comparison of hALEC to Amnion Cells in Human and Macaque Embryos

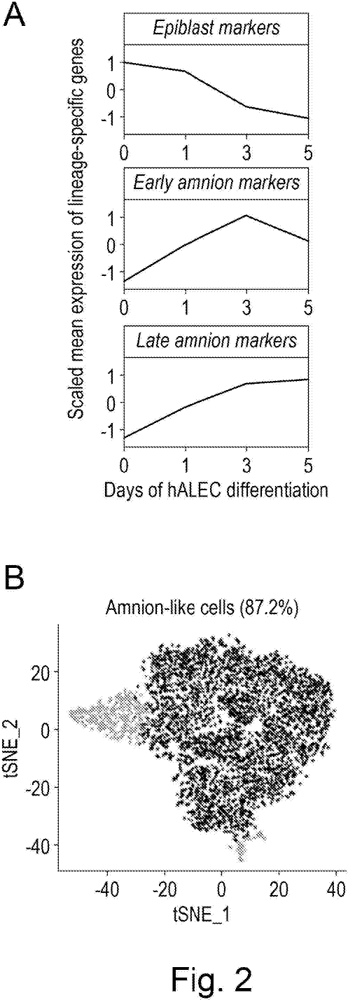

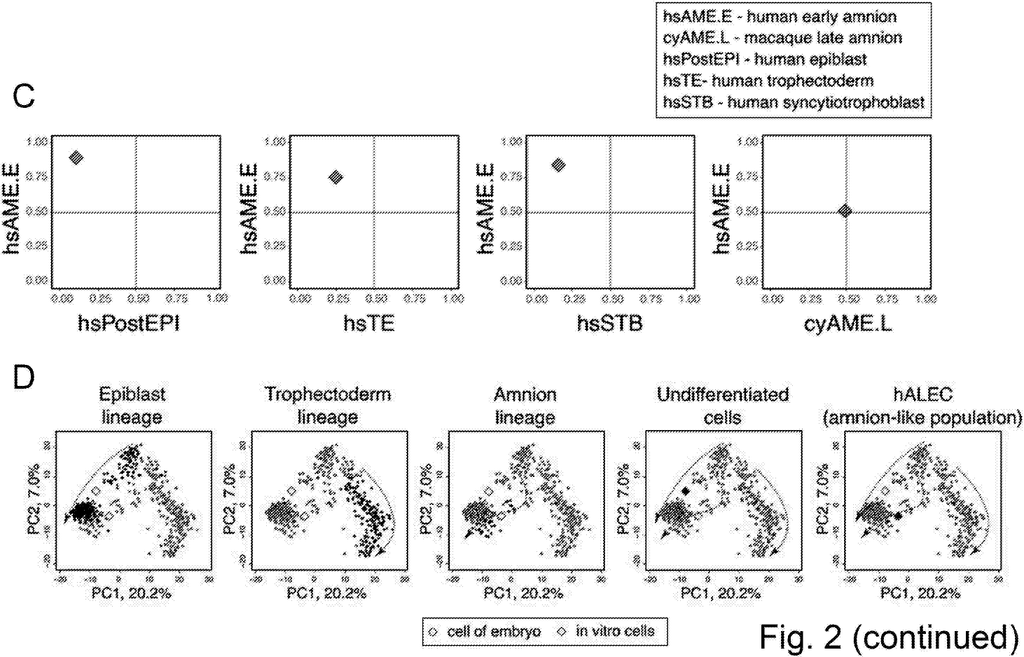

To validate the identity of hALEC, we compared the transcriptome of hALEC (obtained by bulk population and single cell RNA sequencing) to the gene expression profile of ex vivo cultured human and macaque embryos (Xiang et al. Nature 2020; Ma et al. Nature 2019). First, we confirmed that pluripotent epiblast-specific genes are globally downregulated during hALEC differentiation, markers of early amnion are upregulated and peak on day 3, whereas late amnion markers peak on day 5 of hALEC induction (FIG. 2A). Single cell analysis revealed that about 87.2% of hALEC population have characteristics of amnion (FIG. 2B), the average expression of this subpopulation was used for further examination. Analysis of fractions of identity (Gong et al. Bioinformatics 2013) showed that these cells are most similar to amnion cells in embryos (>75% of human amnion identity) and exhibit features of amnion of both human and macaque embryos (FIG. 2C). PCA identified trajectories of epiblast, amnion and trophectoderm progression in embryos (FIG. 2D). As expected, undifferentiated hPSC were positioned on the trajectory of epiblast, whereas hALEC were clearly aligned with amnion cells in PCA. Therefore, our comparison of hALEC to human and macaque embryos evidently validated their identity as amniotic epithelial cells.

### 4. Competence for hALEC Formation Is an Intrinsic Property of Partially Capacitated hPSC

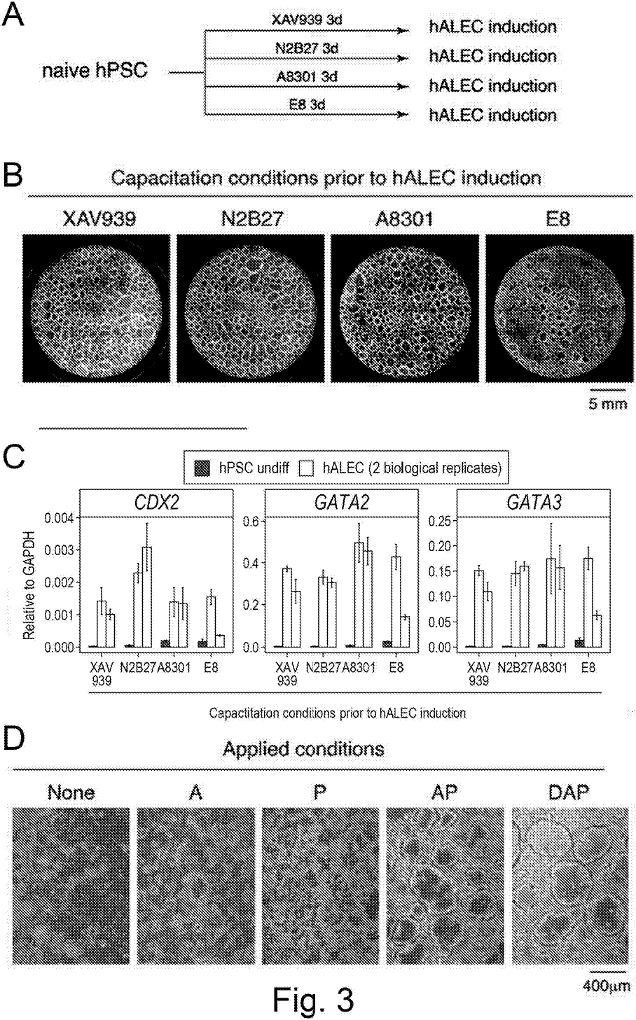

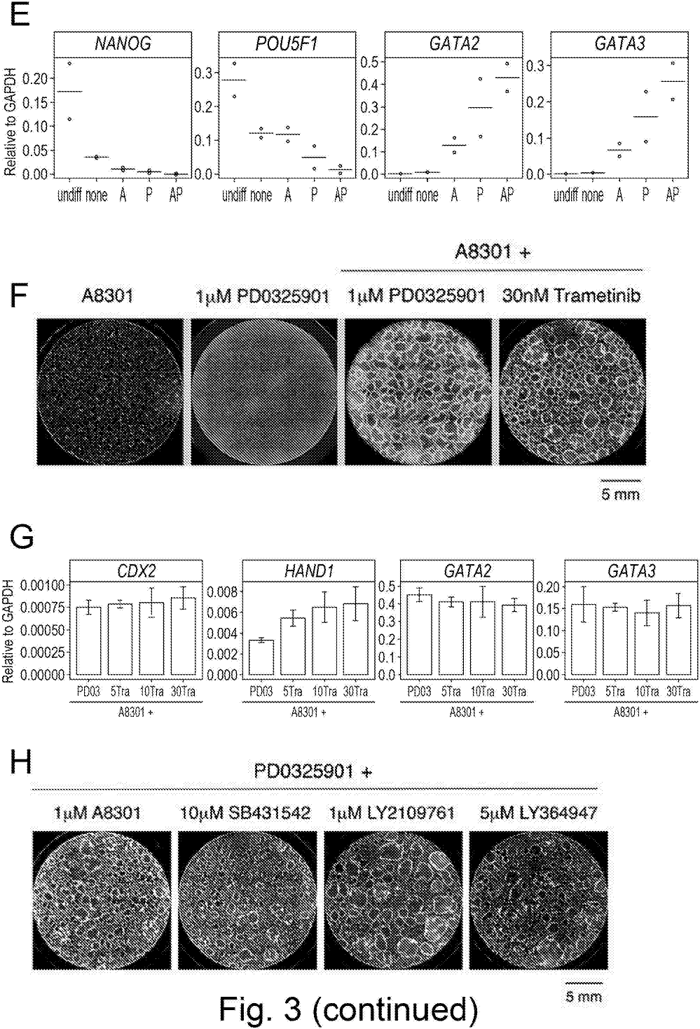

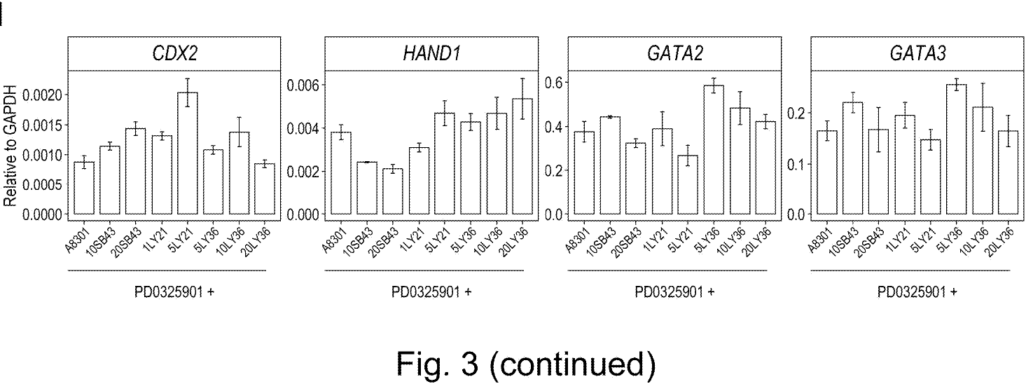

WNT inhibition is beneficial but not essential for the formative transition of hPSC, which is rather guided by their autocrine signalling (Rostovskaya et al. Development 2019). We induced formative transition by the range of conditions: (1) WNT inhibitor XAV939 in N2B27 basal medium; (2) simple withdrawal of the factors (PD0325901, Go6983, XAV939, LIF) for naïve hPSC maintenance from the medium and culturing in basal N2B27 conditions; (3) TGFb inhibitor A8301 in N2B27 basal medium; (4) E8 medium containing TGFb and FGF2 for culturing primed hPSC (Chen et al. Nat Methods 2011); all for 3 days, and then applied AP conditions for differentiation (FIG. 3A). In all conditions hPSCs successfully differentiated to hALEC as indicated by characteristic morphology (FIG. 3B) and markers expression (FIG. 3C). Efficiency of hALEC differentiation was slightly more variable after capacitation in E8 because this condition is known to be suboptimal for cell fitness during the formative transition (Rostovskaya et al. Development 2019), however it was consistently high in the other three conditions. Therefore, the ability to form hALEC capable of self-assembly into spheroids, is an intrinsic property of hPSC that is established during capacitation, rather than a property that is acquired in response to exogenous WNT inhibition.

### 5. Signalling Requirements to Generate hALEC

We next assessed whether the joint inhibition of the TGFb/Activin/Nodal and MAPK pathways is required for the formation of hALEC. We tested this using hPSC after 3-5 days of the capacitation in independent experiments by supplementing their media with either A8301 only (“A”), PD03 only (“P”), neither of the inhibitors (“none”), or both (“AP”). Only the cells treated by both inhibitors were able to efficiently form the 3D bubble-like epithelial structures (FIG. 3D) and consistently upregulated characteristic amnion markers (FIG. 3E). These results demonstrate that the TGFb/Activin/Nodal and MAPK pathways must jointly be inhibited for hALEC differentiation.

Furthermore, we found that successful induction of hALEC can be achieved not only by using a combination of PD0325901 and A8301, but also by alternative inhibitors of MAPK, such as Trametinib (FIGS. 3F and 3G), and TGFb pathway, such as SB431542, LY2109761, LY364947 (FIGS. 3H and 3I). Therefore, hALEC differentiation is induced specifically by inhibition of MAPK and TGFb pathways and not by other effects of PD0325901 and A8301.

The formation of cells expressing a subset of amnion markers has been previously reported after treating conventional primed hPSC with the growth factor BMP4 (Shao et al. Nat Mater 2017; Shao et al. Nat Commun 2017; Zheng et al. Nature 2019). However, this finding is at odds with the timing of events that occur during embryo development. In particular, primed hPSC closely resemble late post-implantation epiblast cells just prior to the onset of gastrulation (Nakamura et al. Nature 2016), a developmental stage that occurs in humans at around 11-12dpf (Carnegie stage, CS, 5c), which is several days after the emergence of the amnion in the implanting embryo at 7dpf (CS 5a) (The Virtual Human Embryo Atlas). Therefore, BMP-dependent route of differentiation reported in the aforementioned works can’t explain the mechanism of formation of amnion and amniotic cavity in primate embryos. Nevertheless, we tested whether BMP signalling was required for hALEC differentiation by adding a selective BMP receptor (ALK-2, -3, -6) inhibitor called LDN193189 to the AP-containing media (a combination referred to as “DAP” hereafter). Blocking BMP signalling did not interfere with hALEC differentiation (FIG. 3D). These results, therefore, show that we have discovered a unique, BMP-independent, route to human amnion differentiation in vitro that has not been reported previously.

### 6. hPSC Gain Transient Competence for Differentiation to hALEC During the Formative Transition

The embryological evidence described above indicates that amnion cells are produced by epiblast during the time of embryo implantation; a stage that also corresponds to when the epiblast cells exit from a naïve pluripotent state, manifested by the loss of the diagnostic naïve markers (Nakamura et al. Nature 2016; Zhou et al. Nature 2019; Xiang et al. Nature 2020). To identify a window during the progression from naïve to primed pluripotency where hPSC have the competence to form amnion, we systematically tested the ability of hPSC at different stages of capacitation to respond to hALEC-inducing cues (FIG. 4A). In these experiments, hALEC differentiation was induced using two alternative media compositions – AP and DAP – in order to assess whether the requirement for BMP pathway activity is altered this window of competence. As an additional control, conventional hESC line H9 (whereby hESC are considered as conventional if they were derived and maintained in the primed state) were also included as a starting cell type. The efficiency of hALEC formation was evaluated visually by their efficiency to form 3D epithelial spheres (FIGS. 4B and 4C), and markers expression (FIG. 4D). In AP-containing conditions, the stage with the highest potential for amnion differentiation was observed when hPSC were induced to hALEC between day 2 and 5 of capacitation. Interestingly, naïve hPSC without prior capacitation (day 0) also produced epithelial spheres in AP conditions, however, the emergence of these spheres was delayed by at least one day and the efficiency of sphere formation was reduced (FIG. 4E), moreover AP-treated naïve hPSC contained a considerable subpopulation of cells that failed to downregulate pluripotency markers such as OCT4. The observed delay in the response by naïve hPSC and their recalcitrance to differentiation suggest that the exit from naïve pluripotency is required prior to hALEC formation. The cells at 1 day of capacitation showed a slightly reduced capacity for hALEC differentiation, as compared to the cells that were capacitated for 2-5 days (data not shown). Notably, if hALEC differentiation was induced at any time point of hPSC capacitation beyond day 0, the spheres that emerged did so simultaneously when comparing between these cell populations and the differentiation showed similar dynamics (results not shown). After day 5 of capacitation, the ability of hPSC to produce hALEC rapidly declined and was lost from day 7-8 onwards. Conventional hESC line H9 did not produce hALEC in these conditions; instead, a large fraction of the cells formed PAX6-positive neuroepithelial cells (FIG. 4G). Thus, during the progression from naïve to primed pluripotency hPSC have a transient competence to differentiate with high efficiency into amnion-like cells.

When testing the role of BMP signalling, we found that hALEC differentiation in DAP-containing media was slightly delayed as compared to AP conditions (result not shown). By day 5 of DAP treatment, however, the cells produced the characteristic spheres with high efficiency and this efficiency was comparable to cells in AP conditions. The time window defined by high competency spanned days 2 to 6, and, therefore, was slightly extended as compared to cells in AP media (FIG. 4C). Moreover, the spheres were readily formed by hPSC even after 7-9 days of capacitation, albeit with a lower efficiency. This extension of the window of competence was observed also in the presence of alternative inhibitors of BMP pathway, such as Dorsomorphin and K02288 (FIG. 4F). The ability to produce hALEC then declined sharply and spheres were not observed when the cells were induced at day 10 of capacitation. Instead, a substantial fraction of induced cells formed PAX6 positive, neuroepithelial cells (FIG. 4G). Primed hPSC also differentiated to the neural lineage and not to amnion when treated with DAP, as confirmed by flow cytometry analysis for PAX6. These results further demonstrate that hALEC differentiation is independent of BMP signalling in our system. Moreover, the inhibition of the BMP pathway potentiated the inhibition of the MAPK and TGFb pathways, at least at the later stages of capacitation, and extended the window of competence.

It is important to note that hPSC strongly downregulate pluripotency markers that define the naïve state, such as KLF4, after 1 day of capacitation, and most of the cells have irreversibly lost the naïve properties by day 3 (Rostovskaya et al. Development 2019). Moreover, hPSC gain the transcriptional signature most similar to primed hPSC only by day 10 of capacitation. Hence, the competence to produce amniotic epithelium encompasses a period of formative transition that occurs after the exit from the naïve state and before the acquisition of the primed state.

1. A method for differentiating pluripotent stem cells into amniotic-like epithelial cells, said method comprising culturing said cells with an inhibitor of the MAPK pathway and an inhibitor of the TGF pathway.

2. The method of claim 1 wherein said amniotic-like epithelial cells form a continuous layer of cells.

3. The method of claim 2 wherein said continuous layer of cells forms a membrane or a 3D structure.

4. The method of any one of claims 1 to 3 wherein said pluripotent stem cells are any one or more of:
i) naïve pluripotent stem cells;
ii) naïve pluripotent stem cells cultured under capacitating conditions;
iii) primed pluripotent stem cells cultured under conditions reverting them to naïve pluripotent stem cells; and/or
iv) pluripotent stem cells representing intermediate states between the naïve and the primed pluripotent states.

5. The method of any one of claims 1 to 4 wherein said pluripotent stem cell is not a primed pluripotent stem cell.

6. The method of any one of claims 1 to 5 wherein said method comprises culturing the pluripotent stem cells with a BMP inhibitor.

7. The method of any one of claims 1 to 6 wherein said MAPK pathway inhibitor is a chemical inhibitor, neutralising antibody, ligand trap, aptamer, antisense nucleotide, protein inhibitor or engineered peptide, or an indirect inhibitor of the MAPK pathway, said MAPK pathway inhibitor targeting any one component of the pathway selected from the list comprising:
receptor tyrosine kinases, Ras, Src, Raf, MEK½, p38 MAP kinases, ERK½; or activators or agonists of AKT and PI3K.

8. The method of any one of claims 1 to 7 wherein said TGF pathway inhibitor is a chemical inhibitor, neutralising antibody, ligand trap, aptamer, antisense nucleotide, protein inhibitor or engineered peptide or an indirect inhibitor of the TGF pathway, said TGF pathway inhibitor targeting any one component of the pathway selected from the list comprising: ligands TGF beta, Activin, Nodal; TGF beta type I receptors TGFBR1, ACVR1, ACVRL1, ACVR1B, ACVR1C; TGF beta type II receptors TGFBR2, ACVR2A, ACVR2B; signal transducers Smad2, Smad3, Smad4; TGF ligand processing enzyme furin.

9. The method of any one of claims 6 to 8 wherein said BMP inhibitor is a chemical inhibitor, neutralising antibody, ligand trap, aptamer, antisense nucleotide, protein inhibitor or engineered peptide or an indirect inhibitor of BMP, said BMP inhibitor targeting any one component of the pathway selected from the list comprising: ligands BMP2, BMP4, BMP7; BMP type I receptors BMPRIA, BMPRIB; BMP type II receptor BMPR2, Smad1, Smad5, Smad8.

10. The method of any preceding claim, wherein the pluripotent stem cells are cultured in suspension.

11. Amniotic-like epithelial cells prepared according to any one of claims 1 to 10.

12. A composition comprising amniotic-like epithelial cells, or an extract or derivative thereof prepared according to any one of claims 1 to 10.

13. The composition of claim 12 which is a pharmaceutical preparation.

14. Use of the cells of claim 11 or the composition of claim 12 or 13 in therapy.

15. Use of the cells of claim 11 or a composition as claimed in claims 12 or 13 for any one or more of:
(a) wound healing and/or tissue repair, optionally skin repair or repair of muscle or connective tissue damage, such as a hernia or pelvic floor repair;
(b) ocular surface repair;
(c) the treatment of burns, ulcers or surgical wounds;
(d) treating diabetes or liver disease;
(e) the treatment of congenital conditions, optionally epidermolysis bullosa;
(f) the treatment of skin necrosis, optionally Stevens Johnson syndrome;
(g) the treatment of urological and/or gynaecological conditions; and/or
(h) as an anti-inflammatory.

16. Amniotic epithelium prepared with cells differentiated according to the method of any one of claims 1 to 10.

17. A membrane prepared with cells differentiated according to the method of any one of claims 1 to 10.

18. A three dimensional structure, such as a hollow sphere or hollow spheroid, prepared with cells differentiated according to the method of any one of claims 1 to 10.

19. The cells of claim 11 or the membrane of claim 17 or structure of claim 18 for use as a research tool.

20. A method as claimed in any one of claims 1 to 10 wherein said cells are human.

21. A cosmetic preparation comprising the cells defined in claim 11 and a cosmetically acceptable carrier.
